(19) United States
(12) Patent Application Publication
(10) Pub. No.: US 2010/0111905 A1
(43) Pub. Date: May 6, 2010

(54) METHODS FOR IMPROVED ENGRAFTMENT FOLLOWING STEM CELL TRANSPLANTATION

(75) Inventor: Andrew E. Balber, Durham, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: ALDAGEN,INC., Durham, NC (US)

(21) Appl. No.: 12/513,882

(22) PCT Filed: Nov. 8, 2007
(86) PCT No.: PCT/US2007/084037

§ 371 Date: Jan. 4, 2010

Related U.S. Application Data
(60) Provisional application No. 60/857,547, filed on Nov. 8, 2006.

(51) Int. Cl. A61K 35/12; A61P 35/00; A61P 19/04

(52) U.S. Cl. 424/93.7

(57) ABSTRACT

The present invention relates to methods repairing, regenerating, and reconstituting tissues by transplanting at least two stem cell populations, wherein the first and the second population of stem cells are introduced into a subject separated by a time interval of about 2 to about 24 hours. The stem cells can be derived from umbilical cord, mobilized peripheral blood, or bone marrow. The cells of at least the second population may be enriched for adult stem and progenitor cells. The methods of the invention are useful in accelerating hematopoeitic recovery in subjects following myeloablation or chemotherapy.

### FIELD OF THE INVENTION

The present invention relates to improved methods of reconstituting, repairing, and regenerating tissue using populations of stem cells enriched for early progenitor cells.

### BACKGROUND OF THE INVENTION

Over the past decade, umbilical cord blood (UCB) transplantation has been shown to be a viable alternative donor stem cell source for hematopoietic cell transplantation in subjects with catastrophic diseases treatable with transplantation therapy. UCB cells can cross partially mismatched HLA barriers without intolerable acute or chronic Graft-versus-Host Disease (GvHD) (Wagner et al. (1996) Blood 88(3):795-802; Rubinstein et al. (1998) N Engl J Med 339(22):1565-1577; Rocha, et al. (2000) N Engl J Med 342(25):1846-1854) Thus, many subjects lacking a sufficiently matched, living related or unrelated bone marrow or adult stem cell donor, can use partially HLA-matched UCB cells for stem cell rescue after myeloablative irradiation and/or chemotherapy. UCB cell dose, expressed per kilogram of recipient body weight, is the best predictor of outcomes after UCB transplantation (Kurtzberg J, et al. (1996) N Engl J Med 335:157-166; Stevens et al. (2002) Blood 100(7):2662-2664). Cell dose thresholds strongly correlating with outcomes have been identified. In subjects receiving lower cell doses, while durable engraftment will ultimately occur, there are significant delays in myeloid and platelet engraftment which, at best, result in longer hospitalization and significant increases in resource utilization and in the worst cases, result in increased early deaths from infection and regimen-related toxicity.

In infants and children weighing <40 kg, it is possible to find a sufficiently matched UCB unit that will deliver a dose of cells critical for successful engraftment (defined as 3×10e7 nucleated cells/kg) within a reasonable time frame in >90% of subjects. In teenagers and adults weighing >40 kg, this is possible 30-50% of the time. Because UCB units contain a relatively fixed number of total nucleated cells, units delivering optimal cell dosing for subjects weighing >70 kg will only be identified <15% of the time. Attempts to increase the dose of cells available for UCBT have included ex vivo expansion and combined unit transplantation. While expansion of UCB cells ex vivo is possible, previous phase I studies of infusion of expanded cells have not resulted in shortening of engraftment times (Jaroscak et al. (2003) Blood 101(12):5061-5067; McNiece et al. (2004) Cytotherapy 6(4):311-317). Likewise, combinations of up to 5 UCB units for a single myeloablative transplant have not shortened time to neutrophil or platelet engraftment.

Several strategies have tried to address ways to increase cells available for transplantation with the intent of shortening the time to neutrophil and/or platelet engraftment. If successful, these approaches would increase the safety of the transplant procedure by lessening regimen-related toxicity. Engraftment after UCBT is a major predictor of overall and event free survival. An intervention that could facilitate engraftment by decreasing time to absolute neutrophil count (ANC) recovery and/or overall probability of engraftment would be advantageous.

### SUMMARY OF THE INVENTION

Methods are provided herein for use in reconstituting, repairing and regenerating tissue in a subject in need thereof by introducing into the subject at least a first and a second population of cells. The first cell population can comprise UCB, bone marrow (BM), mobilized peripheral blood (MPB), or UCB-, BM-, or MPB-derived stem cells, can comprise nucleated cells, stem cells, or mixtures of such cell types with nonviable cells and cell debris such as typically are found in thawed cellular products conventionally used as hematopoietic grafts, or the like, or can comprise agents specifically engineered to facilitate release of the second cell population of stem cells from the liver and/or lungs following introduction into the subject, and to potentiate engraftment of this population of stem cells. The second cell population comprises stem cells isolated from umbilical cord, mobilized peripheral blood, or bone marrow. In some embodiments, the first and/or the second populations of stem cells are substantially enriched for stem and progenitor cells. The methods of the invention are particularly useful in accelerating time to neutrophil and/or platelet engraftment and immune reconstitution following myeloablative therapy where the cell populations are administered through a central intravenous line. The methods can be used to treat patients with malignant disorders, genetic disorders, immune disorders, or used to repair tissue following damage.

### DETAILED DESCRIPTION OF THE INVENTION

### I. Overview

Stem and progenitor cells (SPC) reproduce and maintain developmental potential until specific biological signals induce the cells to differentiate into a specific cell type or tissue type. Adult stem and progenitor cells (ASPC) are small populations of SPC that remain in tissues of an organism following birth and are continuously renewed during a lifetime. In vitro colony assays have demonstrated that bone marrow (BM), mobilized peripheral blood (MPB), and umbilical cord blood (UCB), all contain a variety of ASPC. Bone marrow is particularly rich in multipotential ASPC.

As used herein, “stem cell” refers to a cell with the capability of differentiation and self-renewal, as well as the capability to regenerate tissue. Although stem cells are described mostly with respect to using umbilical cord blood stem cells in the present application, the invention is not limited to such and may include adult stem cells of other origin, including but not limited to liver stem cells, pancreatic stem cells, neuronal stem cells, bone marrow stem cells, peripheral blood stem cells, umbilical cord blood stem cells or mixtures thereof. In some embodiments, the present invention excludes the use of embryonic stem cells. Further, the invention is not limited to transplantation of any particular stem cell obtained from any particular source, but may include stem cells from “multiple stem cell sources” in mixture with one another. Thus, mesenchymal stromal cells may be used in cotransplantation of the stem cells obtained from single or multiple stem cell sources to increase the amount of engraftment.

As used herein, “engraftment” and “in vivo regeneration” refer to the biological process in which implanted or transplanted stem cells reproduce themselves and/or produce differentiated cell progeny in a host organism, and/or replace lost or damaged cells in the host.

Allogeneic cell therapies are used to treat a variety of diseases or pathological conditions. Allogeneic cell therapy is an important curative therapy for several types of malignancies and viral diseases. Allogeneic cell therapy involves the infusion or transplant of cells to a subject, whereby the infused or transplanted cells are derived from a donor other than the subject. As used herein, the term “derive” or “derived from” is intended to obtain physical or informational material from a cell or an organism of interest, including isolation from, collection from, and inference from the organism of interest.

Types of allogeneic donors that have been utilized for allogeneic cell therapy protocols include: human leukocyte antigen (HLA)-matched siblings, matched biologically unrelated donors, partially matched biologically related donors, biologically related umbilical cord blood donors, and biologically unrelated umbilical cord blood donors. The allogeneic donor cells are usually obtained by bone marrow harvest, collection of peripheral blood or collection of placental cord blood at birth.

The methods of the present invention encompass the administration or introduction of two cell preparations (or “populations”), wherein the administration of each is separated in time so as to accelerate hematopoiesis. “Administration” or “introduction” refers to the intravenous introduction of the cell populations described herein into a subject. In some embodiments, the administration of the two cell preparations follows myeloablative therapy. The method of administration for each of the cell populations need not be the same. For instance, the first cell population can be administered by infusion, and the second cell population can be administered by injection.

For the purposes of the present invention, one cell preparation is referred to as the “first cell population” and the other cell preparation is referred to as the “second cell population” or “supplement cell population.” The second cell population is administered to a subject no more than about 1 hour, no more than about 1.5 hours, about 2 hours, about 2.5 hours, about 3 hours, about 3.5 hours, about 4 hours, about 4.5, about 5, about 5.5, about 6, about 6.5, about 7, about 7.5, about 8, about 8.5, about 9, about 9.5, about 10, about 11, about 12, about 13, about 14, about 15, about 16, about 17, about 18, about 19, about 20, about 21, about 22, about 23, or no more than about 24 hours after the first cell population.

The first cell population can comprise UCB, BM, MPB, or UCB-, BM-, or MPB-derived stem cells, can comprise nucleated cells, stem cells, or mixtures of such cell types with nonviable cells and cell debris such as typically are found in thawed cellular products conventionally used as hematopoietic grafts, or the like. Additionally, the first population can comprise agents specifically engineered to facilitate release of the second cell population of stem cells from the liver and/or lungs following introduction into the subject. Such agents may include any molecule capable of specifically or nonspecifically binding endothelial cell receptors involved in extravasation of stem cells, or any molecule capable of blocking phagocytosis by reticuloendothelial macrophages, as discussed below (for example, colloidal iron, anti-selectin antibodies, etc) The second cell population comprises any cell population capable of engraftment. A cell population that is “capable of engraftment” is one that results in the growth and function of donor cells in a recipient subject. In one embodiment, the cell population does not comprise embryonic stem cells. In another embodiment, the second cell population comprises UCB-, BM-, or MPB-derived cells that have been purified and/or enriched for primitive stem cell populations. The first cell population and the second cell population may be obtained or derived from different sources of cells. For example, the first cell population can be derived from umbilical cord, whereas the second cell population can be derived from bone marrow or MPB. Alternatively, the first and second cell populations can be derived from the same source (e.g, UCB, MPB, or BM) from the same donor. In another embodiment, the first and second cell populations can be derived from different sources, provided, however, that donor cells are selected or manipulated by conventional methods to minimize potential for the development of graft versus host disease (GvHD) and/or graft rejection (e.g., by partial or full HLA-matching). Where the first and second cell populations are derived from the same donor, the UCB, BM, or MPB collected from the donor can be apportioned into about an 80%/20%, about a 75%/25%, about a 60%/40%, about a 65%/35%, about a 60%/40%, about a 55%/45%, or about a 50%/50% split for the first and second cell populations, respectively. This split can be an apportionment of one batch of cells collected at a particular time (e.g., a single bag of blood or blood product collected from the donor, split according to the parameters above), or it can be an apportionment the total amount of blood or blood product collected from the donor over a period of time (e.g., pooled batches from the donor).

The number of nucleated cells required for each infusion depends on the composition of the first cell population. For example, if the first cell population contains a sufficient number of cells to facilitate release of the second cell population of stem cells from the liver and/or lungs following introduction into the subject, the second cell population may contain fewer stem cells. Alternatively, or in addition, if the second cell population contains a sufficient number of stem cells to engraft, the first cell population may not need abundant or even any ASPC. Optimal doses of both first and second cell populations are discussed elsewhere herein.

While not being bound to any particular theory or mechanism of action, it is thought that the infusion of the initial cell preparation may engage receptors and the like in the liver and lungs that tend to sequester infused cells and limit the number of viable stem cells that home to the bone marrow for engraftment. Current methods for infusion of graft material in transplantation all involve intravenous infusion through a central line. This route brings infused material immediately to the pulmonary circulation. Much of the material infused is retarded by the small microvasculature in the lungs where the pulmonary venous capillaries join the pulmonary arterioles in the area around the alveoli. This area is lined with pulmonary macrophages that can remove particulate material and dying cells, both of which are common in unpurified graft preparations derived from thawed UCB, BM, and MPB. Flow of material through this microvasculature will be slowed by accumulation of debris and dead cells in the graft and this, in turn, will delay or inhibit the ability of stem/progenitor cells to leave the lungs for the circulation (reviewed in Hall (1985) Immunol Today 6(5):149-152, herein incorporated by reference in its entirety), likely by phagocytosis in the reticuloendothelial system.

The reticuloendothelial system (RES), consisting of fixed macrophages of the liver, spleen, lungs, and bone marrow, provides the host with a variety of immunological defenses including protection against infection, neoplasia surveillance, and recognition of foreign antigens (Gilbreath et al. (1985) J Immunol 134:6420; Saba (1981) Arch Int Med 126:1031). The latter function of processing antigens enhances the host's capacity to reject allogeneic tissue and organ transplants, and has stimulated interest in suppression of the RES to modulate the rejection of transplanted tissue.

Pretreatment of animals with dextran sulfate and carbon resulted in a decrease in uptake of liposomes by phagocytic cells of the RES, apparently as a result of the saturation of the uptake capacity of the cells of the RES (Souhami et al. (1981) Biochim Biophys Acta 674:354).

In addition to influencing RES activity, while not being bound by any particular theory or mechanism, cells and cell debris in the first population may, by continuing to occupy endothelial receptors for ligands expressed on therapeutically active cells in the second population, limit adhesion of cells in the second population to the pulmonary endothelium. In essence, the first population hides or “masks” the endothelium from the cells in the second population that express similar ligands. The cells in the second population are therefore more free to leave the lungs, travel through the blood to the bone marrow, and compete for important microenvironmental marrow niches for successful engraftment.

While others have described the use of an initial and a supplemental infusion of cell preparations, therapeutic response has not been improved when compared to subjects receiving only a first cell population (Fernandez et al. (2003) Exp Hematol. 31(6):535-44; Shpall et al. (2002) Biol Blood Marrow Transplant. 8(7):368-376). These workers describe a protocol whereby the second cell population was administered several days or weeks following the administration of the first cell population. In contrast, the present method contemplates administration of the second cell population within 24 hours of the first population. While not being bound by any particular theory or mechanism, it is possible that the time delay between the initial and second cell population is too long to appreciate any beneficial effects mediated by blocking RES activity and adhesion to the endothelium.

Thus, the compositions of the present invention comprising a first and a second population of cells are useful in a method of reconstituting blood tissue or other stem and progenitor cell function, wherein the method comprises introducing the second population of cells into a subject in need thereof between 2 and 24 hours after the first population of cells. In these and other embodiments, at least the second population is capable of engraftment. In specific embodiments, at least the second population is enriched for ASPC.

### II. Indications

The cell populations described herein can be used for a wide variety of treatment protocols in which a tissue or organ of the body is augmented, repaired or replaced by the engraftment, transplantation or infusion of these cell populations. As used herein, “treatment” is an approach for obtaining beneficial or desired clinical results (i.e., “therapeutic response”). For purposes of this invention, beneficial or desired clinical results include, but are not limited to, alleviation of symptoms, diminishment of extent of disease, stabilization (i.e., not worsening) of disease, delay or slowing of disease progression, amelioration or palliation of the disease state, and remission (whether partial or total), whether detectable or undetectable. “Treatment” can also mean prolonging survival as compared to expected survival if not receiving treatment or receiving different treatment (i.e., only a single dose of cells, or multiple doses of cells spaced greater than 24 hours apart, or some other treatment not encompassed herein). “Treatment” refers to both therapeutic treatment and prophylactic or preventative measures. Those in need of treatment include those already with the disorder as well as those in which the disorder is to be prevented. “Alleviating” a disease means that the extent and/or undesirable clinical manifestations of a disease state are lessened and/or the time course of the progression is slowed or shortened, as compared to a situation without treatment or a different treatment. Typically, the “treatment” entails administering additively effective stem and progenitor cells to the subject to regenerate tissue (particularly hematopoietic cells).

The cell populations useful in the methods described herein may be utilized in a variety of contexts. In one embodiment, the cells may be administered to subjects who have decreased hematologic function resulting from one or more diseases, treatments, or a combination thereof, to accelerate hematologic recovery.

For example, the methods of the invention are useful for the treatment of patients having: diseases resulting from a failure or dysfunction of normal blood cell production and maturation, hyperproliferative stem cell disorders, aplastic anemia, pancytopenia, thrombocytopenia, red cell aplasia, Blackfan-Diamond syndrome due to drugs, radiation, or infection, idiopathic; hematopoietic malignancies, including acute lymphoblastic (lymphocytic) leukemia, chronic lymphocytic leukemia, acute myelogenous leukemia, chronic myelogenous leukemia, acute malignant myelosclerosis, multiple myeloma, polycythemia vera, agnogenic myelometaplasia, Waldenstrom's macroglobulinemia, Hodgkin's lymphoma, non-Hodgkins's lymphoma; immunosuppression in subjects with malignant, solid tumors, including malignant melanoma, carcinoma of the stomach, ovarian carcinoma, breast carcinoma, small cell lung, carcinoma, retinoblastoma, testicular carcinoma, glioblastoma, rhabdomyosarcoma, neuroblastoma, Ewing's sarcoma, lymphoma; autoimmune diseases, rheumatoid arthritis, diabetes type I, chronic hepatitis, multiple sclerosis, and systemic lupus erythematosus; genetic (congenital) disorders, anemias, familial aplastic, Fanconi's syndrome, Bloom's syndrome, pure red cell aplasia (PRCA), dyskeratosis congenital, Blackfan-Diamond syndrome, congenital dyserythropoietic syndromes I-IV, MPS I, MPS II, MPS III, MPS IV, MPS V, Infantile Krabbe disease, adrenoleukodystrophy, metachromatic leukodystrophy, Tay Sachs disease, Chwachmann-Diamond syndrome, dihydrofolate reductase deficiencies, formamino transferase deficiency, Lesch-Nyhan syndrome, congenital spherocytosis, congenital elliptocytosis, congenital stomatocytosis, congenital Rh null disease, paroxysmal nocturnal hemoglobinuria, G6PD (glucose-6-phosphate dehydrogenase), variants 1,2,3, pyruvate kinase deficiency, congenital erythropoietin sensitivity, deficiency, sickle cell disease and trait, thalassemia alpha, beta, gamma met-hemoglobinemia, congenital disorders of immunity, severe combined immunodeficiency disease, (SCID), bare lymphocyte syndrome, ionophore-responsive combined, immunodeficiency, combined immunodeficiency with a capping abnormality, nucleoside phosphorylase deficiency, granulocyte actin deficiency, infantile agranulocytosis, Gaucher's disease, adenosine deaminase deficiency, Kostmann's syndrome, reticular dysgenesis, congenital leukocyte dysfunction syndromes; osteopetrosis, myelosclerosis, acquired hemolytic anemias, acquired immunodeficiencies, disorders involving disproportions in lymphoid cell sets and impaired immune functions due to aging phagocyte disorders, Kostmann's agranulocytosis, chronic granulomatous disease, Chediak-Higachi syndrome, neutrophil actin deficiency, neutrophil membrane GP-180 deficiency, metabolic storage diseases, mucopolysaccharidoses, mucolipidoses, miscellaneous disorders involving immune mechanisms, Wiskott-Aldrich Syndrome, and alpha 1-antitrypsin deficiency.

It has also been shown that the hematologic toxicity sequelae observed with multiple cycles of high-dose chemotherapy is relieved by conjunctive administration of autologous hematopoietic stem cells. Thus, the present method is useful for diseases for which reinfusion of stem cells following myeloablative chemotherapy has been described including acute leukemia, Hodgkin's and non-Hodgkin's lymphoma, neuroblastoma, testicular cancer, breast cancer, multiple myeloma, thalassemia, and sickle cell anemia (Cheson et al. (1989) Ann. Intern. Med. 30 110:51; Wheeler et al. (1990) J. Clin. Oncol. 8:648; Takvorian et al. (1987) N. Engl. J. Med. 316:1499; Yeager, et al. (1986) N. Eng. J. Med. 315:141; Biron et al. (1985) In Autologous Bone Marrow Transplantation: Proceedings of the First International Symposium, Dicke et al., eds., p. 203; Peters (1985) ABMT, id. at p. 189; Barlogie, (1993) Leukemia 7:1095; Sullivan, (1993) Leukemia 7:1098-1099).

Most chemotherapy agents used to target and destroy cancer cells act by killing all proliferating cells, i.e., cells going through cell division. Since bone marrow is one of the most actively proliferating tissues in the body, hematopoietic stem cells are frequently damaged or destroyed by chemotherapy agents and in consequence, blood cell production is diminishes or ceases. Thus, the present invention is useful for improving myeloablative transplant outcomes by accelerating platelet and neutrophil engraftment following chemotherapy.

### III. Source of Cell Preparations

The methods of the invention generally encompass the use of allogeneic stem cell therapy. Allogeneic cell therapy is an important curative therapy for several types of malignancies and viral diseases. Allogeneic cell therapy involves the infusion or transplant of cells to a subject, whereby the infused or transplanted cells are derived from a donor other than the subject. Types of allogeneic donors that have been utilized for allogeneic cell therapy protocols include: HLA-matched siblings, matched unrelated donors, partially matched family member donors, related umbilical cord blood donors, and unrelated umbilical cord blood donors. The allogeneic donor cells are usually obtained by bone marrow harvest, collection of peripheral blood or collection of placental cord blood at birth.

Allogeneic cells preferably are chosen from human leukocyte antigen (HLA)-compatible donors. Generally, HLA-compatible lymphocytes may be taken from a fully HLA-matched relative such as a parent, brother or sister. However, donor lymphocytes may be sufficiently HLA-compatible with the recipient to obtain the desired result even if a sibling donor is single-locus mismatched. If a donor is unrelated to the recipient, preferably the donor lymphocytes are fully HLA matched with the recipient. In one embodiment, the cells will be obtained from a donor that is HLA-matched at 6/6 loci. In another embodiment, the cells will be obtained from a donor that is HLA-matched at 5/6 loci. In yet another embodiment, the cells will be obtained from a donor that is HLA-matched at 4/6 loci. Mismatches at the A locus are preferred over mismatches at the B locus, which are preferred over mismatches at the DR locus. In various embodiments utilizing UCB, it may not be necessary to HLA-type the cells prior to administration

Thus, in one embodiment, the invention provides a method of treating an individual comprising administering to the individual a first and a second population of cells collected from at least one donor. “Donor” in this context means an adult, child, infant, or a placenta. In another embodiment, the method comprises administering to the individual a first and/or a second population of cells that has been collected from a plurality of donors and pooled. Alternatively, the first and the second population of stem cells may be taken from multiple donors separately, and administered separately, e.g., one or more donors is used for the first cell population, and one or more of the same or different donors is used for the second cell population.

While the methods described herein generally describe allogeneic transplantation of donor cells, it is contemplated, for various embodiments, that autologous transplantation of host (or “subject”) cells be performed. It is further contemplated that the first cell population can be allogeneic while the second cell population is autologous, or that the first cell population can be autologous while the second cell population is allogeneic. It may be particularly advantageous to utilize UCB, BM, or MPB derived cells from the subject for the repair of tissues other than hematopoietic tissues (or, in some embodiments, including hematopoietic tissues), for example, for tissue regeneration and repair following damage or disease.

### IV. Collection Methods

Umbilical cord blood may be collected in any medically or pharmaceutically-acceptable manner. Various methods for the collection of cord blood have been described. See, e.g., Coe, U.S. Pat. No. 6,102,871; Haswell, U.S. Pat. No. 6,179,819 B1. UCB may be collected into, for example, blood bags, transfer bags, or sterile plastic tubes. UCB or stem cells derived therefrom may be stored as collected from a single individual (i.e., as a single unit) for administration, or may be pooled with other units for later administration.

Bone marrow may be obtained by aspiration from most preferably the posterior iliac crest. Progenitor cells may also be isolated from a donor or subject by treatment with filgrastim (granulocyte colony-stimulating factor, or G-CSF [Neupogen, Thousand Oaks, Calif.]), which will mobilize peripheral-blood progenitor cells (Brugger et al. (1993) Br J Haematol. 84(3):402-7). The cells may be collected in a leukapheresis as described in Brugger (1993).

If frozen, the cells are transferred to an appropriate cryogenic container and the container decreased in temperature to generally from −120° C. to −196° C. and maintained at that temperature. When needed, the temperature of the cells (i.e., the temperature of the cryogenic container) is raised to a temperature compatible with introduction into the subject (generally from around room temperature to around body temperature, e.g., from about 20° C. to about 37.6° C., inclusive), and the cells are introduced into a subject as discussed below.

### V. Enrichment of Stem Cells

In various embodiments of the present invention, at least the second cell population is enriched for ASPC. In some embodiments, both the first and the second cell population are enriched using one or more (in any combination) of the enrichment methods described herein or known in the art. As used herein the terms “enriched” or “enrichment,” when used in conjunction with the number of ASPC in a cell population, means that the total number of ASPC is constant or increased in proportion to the total number of cells in the cell population when compared to an unmanipulated cell population, or compared to a population that has been manipulated by a manner not disclosed herein.

Using a combination of cell surface markers and other markers such as intracellular enzymes and the light scattering properties of the cells, cell populations of the invention can be advantageously “tailored” for particular therapeutic uses by sorting or isolating the cells based on these properties. Also, because some of these unique stem cell populations represent certain cell lineages, the populations can be used to selectively reconstitute certain cell lineages in vivo. Stem cells that give rise to hematopoietic lineages can be used to increase the concentration and potency of stem cell grafts and, thereby, decrease toxicity. Advantageously, these cells can be sorted from autologous bone marrow and peripheral blood, thus further reducing the chance of rejection and increasing the efficacy of stem cell grafts. Stem cells that give rise to mesenchymal tissues such as bone, nerves, oligodendrocytes, muscles, vasculature, bone marrow stroma, and dermis can be used to repair or replace diseased or damaged tissues. By “sorted” or “isolated” is intended stem cells collected from a mammal and contacted with a cell marker, including but not limited to an antibody (conjugated or unconjugated), a fluorescent marker, an enzymatic marker, a dye, a stain, and the like.

By “cell surface marker” is intended a protein expressed on the surface of a cell, which is detectable via specific antibodies. Cell surface markers that are useful in the invention include, but are not limited to, the CD (clusters of differentiation) antigens CD1a, CD2, CD3, CDS, CD7, CD8, CD10, CD13, CD14, CD16, CD19, CD29, CD31, CD33, CD34, CD35, CD38, CD41, CD45, CD56, CD71, CD73, CD90,

CD105, CD115, CD117, CD124, CD127, CD130, CD133, CD138, CD144, CD166, HLA-A, HLA-B, HLA-C, HLA-DR, VEGF receptor 1 (VEGF-R1), VEGF receptor-2 (VEGF-R2), and glycophorin A. By “intracellular marker” is intended expression of a gene or gene product such as an enzyme that is detectable. For example, aldehyde dehydrogenase (ALDH) is an intracellular enzyme that is expressed in most hematopoietic stem cells. It can be detected via flow cytometry by using fluorescent substrates (see, for example, U.S. Pat. Nos. 5,876,956, 6,627,759, and 6,991,897, and U.S. patent application Ser. Nos. 11/247,764 and 10/589,173, each of which is herein incorporated by reference in its entirety).

Populations may be further enriched based on light scattering properties of the cells based on side scatter channel (SSC) brightness and forward scatter channel (FSC) brightness. By “side scatter” is intended the amount of light scattered orthogonally (about 90° from the direction of the laser source), as measured by flow cytometry. By “forward scatter” is intended the amount of light scattered generally less than 90° from the direction of the light source. Generally, as cell granularity increases, the side scatter increases and as cell diameter increases, the forward scatter increases. Side scatter and forward scatter are measured as intensity of light. Those skilled in the art recognize that the amount of side scatter can be differentiated using user-defined settings. By the terms “low side scatter” and “SSC^{lo}” is intended less than about 50% intensity, less than about 40% intensity, less than about 30% intensity, or even less intensity, in the side scatter channel of the flow cytometer. Conversely, “high side scatter” or “SSC^{hi}” cells are the reciprocal population of cells that are not SSC^{lo}. Forward scatter is defined in the same manner as side scatter but the light is collected in forward scatter channel. Thus, the embodiments of the invention include selection of stem cell populations based on combinations of cell surface markers, intracellular markers, and the light scattering properties of cells obtained from a stem cell source.

At least the second cell population of stem cells useful in the methods disclosed herein can comprise ASPC that can be sorted based on the positive expression of markers. In some embodiments, both the first and the second population of cells are sorted based on the positive expression of markers. By “positive for expression” is intended the marker of interest, whether intracellular or extracellular, is detectable in or on a cell using any method, including, but not limited to, flow cytometry. The terms “positive for expression,” “positively expressing,” “expressing,” “⁺” used in superscript, and “^{pos}” used in superscript are used interchangeably herein. By “negative for expression” is intended the marker of interest, whether intracellular or extracellular, is not detectable in or on a cell using any particular method, including but not limited to flow cytometry. The terms “negative for expression,” “negative expressing”, “not expressing,” “⁻” used in superscript, and “^{neg}” used in superscript are used interchangeably herein.

By “^{br}” used in superscript is intended positive expression of a marker of interest that is brighter as measured by fluorescence (using for example FACS) than other cells also positive for expression. Those skilled in the art recognize that brightness is based on a threshold of detection. Generally, one of skill in the art will analyze the negative control sample first, and set a gate (bitmap) around the population of interest by FSC and SSC and adjust the photomultiplier tube voltages and gains for fluorescence in the desired emission wavelengths, such that 97% of the cells appear unstained for the fluorescence marker with the negative control. Once these parameters are established, stained cells are analyzed and fluorescence recorded as relative to the unstained fluorescent cell population. As used herein the term “bright” or “^{br}” in superscript is intended greater than about 20-fold, greater than about 30-fold, greater than about 40-fold, greater than about 50-fold, greater than about 60-fold, greater than about 70-fold, greater than about 80-fold, greater than about 90-fold, greater than about 100-fold, or more, increase in detectable fluorescence relative to unstained control cells. Conversely, as used herein, the terms “dim” or “^{dim}” in superscript is intended the reciprocal population of those defined as “bright” or “^{br}”.

In some embodiments, at least the second population of cells can be enriched based on expression of markers. For example, without limitation, expression of CD34, a highly glycosylated type I transmembrane protein, or CD133, a pentaspan transmembrane glycoprotein, by some cells has been associated with stem and progenitor cell activities, and cells expressing either or both of these antigens can be isolated by cell sorting for therapeutic use. In other embodiments, both the first and the second population of cells are sorted based on the positive expression of such markers.

In some embodiments, at least the second population of cells can be enriched based on the lack of expression of a cell surface marker (i.e., the enriched population is “substantially free” of cells expressing these markers). For example, without limitation, cells that express CD45 are typically in the hematopoietic lineage, and these cells can be removed by cell sorting or other methods to produce cell populations enriched in non-hematopoietic elements, such as endothelial progenitor cells, for therapeutic purposes. In other embodiments, both the first and the second populations of cells can be enriched based on the lack of expression of these cell surface markers.

In some embodiments at least 10%, 20%, or 30% of the ASPC within at least the second cell population useful in the methods of the invention express the cell markers of interest; in other embodiments at least 40%, 50%, or 60% of the ASPC within at least the second cell population express the cell markers of interest; in yet other embodiments at least 70%, 80%, or 90% of the ASPC within at least the second cell population express the cell markers of interest; in still other embodiments at least 95%, 96%, 97%, 98%, 99%, or even 100% of the ASPC within at least the second cell population express the cell markers of interest. By “substantially free” is intended less than about 5%, 4%, 3%, 2%, 1%, or even 0% of the cells in the population express the marker of interest. While the use of purified cell populations from umbilical cord blood is specifically exemplified herein, the use of such cells from other sources, including bone marrow, peripheral blood, and fetal liver, is also contemplated.

Selective methods known in the art and described herein can be used to further enrich ASPC. Commonly, sources of ASPC are reacted with monoclonal antibodies, and subpopulations of cells expressing cognate cell surface antigens are either positively or negatively selected with immunomagnetic beads by complement mediated lysis, agglutination methods, or fluorescence activated cell sorting (FACS). The functional attributes of the resulting subpopulations with a defined cell surface phenotype are then determined using a colony-forming assay. Once the phenotype of cells that do and do not have ASPC activity is known, these methods can be used to select appropriate ASPC for therapeutic transplantation.

If desired, a large proportion of terminally differentiated cells may be removed by initially using a negative selection separation step. For example, magnetic bead separations may be used initially to remove large numbers of lineage committed cells from at least the second population of cells. Desirably, at least about 80%, usually at least about 70%, of the total cells will be removed.

Procedures for cell separation may include, but are not limited to, positive or negative selection by means of magnetic separation using antibody-coated magnetic beads, affinity chromatography, cytotoxic agents joined to a monoclonal antibody or used in conjunction with a monoclonal antibody, including, but not limited to, complement and cytotoxins, and “panning” with antibody attached to a solid matrix, e.g., plate, elutriation, or any other convenient technique.

Techniques providing accurate cell separation include, but are not limited to, flow cytometry, which can have varying degrees of sophistication, e.g., a plurality of color channels, low angle and obtuse light scattering detecting channels, impedance channels, and the like. The antibodies for the various dedicated lineages may be illuminated by different fluorochromes. Fluorochromes that may find use in a multicolor analysis include phycobiliproteins, e.g., phycoerythrin and allophycocyanins; fluorescein; and Texas red. The cells of at least the second population of cells may also be selected against dead cells, by employing dyes that selectively accumulate in dead cells (e.g., propidium iodide and 7-aminoactinomycin D (7-AAD)). Preferably, the cells are collected in a medium comprising about 2% fetal calf serum (FCS) or 0.2% bovine serum albumin (BSA). See, for example, Fallon et al. (2003) Br. J. Haematol. 121:1, herein incorporated by reference.

Other techniques for positive selection may be employed, which permit accurate separation, such as affinity columns, and the like. The method of choice should permit the removal of the non-progenitor cells to a residual amount of less than about 20%, less than about 15%, less than about 10%, or less than about 5% of the desired population of at least the second population of cells.

Regardless of the isolation procedure used for the enrichment step(s), the resulting cells of at least the second population have therapeutically important properties. In one embodiment, at least the second cell population useful in the methods of the invention comprises ALDH^{br} cells. ALDH^{br} cells express high levels of the enzyme aldehyde dehydrogenase and give low side scatter signals in flow cytometric analysis. The various properties of ALDH^{br} cell populations and methods of obtaining them are well known in art. See, for example, U.S. Pat. No. 6,537,807; U.S. Pat. No. 6,627,759; Storms et al. (1999) Proc. Natl. Acad. Sci USA 96:9118; PCT Publication No. WO2005/083061; Storms et al. (2005) Blood 106(1):95-102; and, Hess et al. (2004) Blood 104(6):1648-55, each of which is herein incorporated by reference in their entirety.

These ALDH^{br} cells can be used to generate any cell of the hematopoietic lineage, including, but not limited to, myeloid cells (such as platelets, megakaryocytes, and red bloods cells) and lymphoid cells (such as T cells, B cells, NK cells, and antigen presenting cells). These cells are also capable of generating any cell of the hematopoietic lineage, including, but not limited to, myeloid cells (such as platelets, megakaryocytes, and red bloods cells) and lymphoid cells (such as T cells, B cells, NK cells, and antigen presenting cells).

Mesenchymal stem cells (MSC) have been characterized using panels of antibodies much like hematopoietic stem cells. MSC generally lack expression of CD14, CD34, and CD45. MSC are generally positive for CD105 and CD73. Other markers used by researchers to identify cultured mesenchymal cells include positive expression of such markers as CD29, Thy-1 (CD90), CD115, CD144, CD166, and HLA-A, B, or C. Functionally, MSC can be tested in vitro for their ability to differentiate into adipogenic, osteogenic, myogenic, and chondrogenic cell colonies.

In other embodiments, at least the second cell population useful in the methods of the invention isolated from UCB, for example, is ALDH^{br}. In yet other embodiments, at least the second cell population isolated from bone marrow and mobilized peripheral blood, for example, is ALDH^{br}

Other methods of stem cell purification or concentration can include the use of techniques such as counterflow centrifugal elutriation, equilibrium density centrifugation, velocity sedimentation at unit gravity, immune rosetting and immune adherence, and T lymphocyte depletion.

Enriched ASPC can be manipulated ex vivo or expanded to promote development of specific types of cells upon subsequent transplantation. For example, UCB cells have been expanded in order to try to hasten neutrophil, erythroid, and platelet engraftment after allogeneic transplantation. Techniques for ex vivo expansion are well described in the art. See, for example, McNiece and Briddle (2001) Exp. Hematol. 29:3; McNiece et al. (2000) Exp. Hematol. 28:1186; Jaroscak et al. (2003) Blood 101:5061. This invention forsees that such expanded or manipulated cell populations, which in fact comprise cell populations selected through cultivation, may also be used for the first or second cell populations.

### VI. Administration

The cell populations useful in the methods of the present invention have application in a variety of therapies and diagnostic regimens. They are preferably diluted in a suitable carrier such as buffered saline before administration to a subject. The cells may be administered in any physiologically acceptable vehicle. Cells are conventionally administered intravascularly by injection, catheter, or the like through a central line to facilitate clinical management of a patient. This route of administration will deliver cells on the first pass circulation through the pulmonary vasculature. Usually, at least about 1×10⁵ cells/kg and preferably about 1×10⁶ cells/kg or more will be administered in the first cell population of cells, or in the combination of the first and second cell population. See, for example, Sezer et al. (2000) J. Clin. Oncol. 18:3319 and Siena et al. (2000) J. Clin. Oncol. 18:1360 If desired, additional drugs such as 5-fluorouracil and/or growth factors may also be co-introduced. Suitable growth factors include, but are not limited to, cytokines such as IL-2, IL-3,IL-6, IL-11, G-CSF, M-CSF, GM-CSF, gamma-interferon, and erythropoietin. In some embodiments, the cell populations of the invention can be administered in combination with other cell populations that support or enhance engraftment, by any means including but not limited to secretion of beneficial cytokines and/or presentation of cell surface proteins that are capable of delivering signals that induce stem cell growth, homing, or differentiation. In these embodiments, less than 100% of the second cell population comprises enriched stem cells.

In some embodiments, first and/or second population of stem cells may be conditioned by the removal of red blood cells and/or granulocytes after it has been frozen and thawed using standard methods.

The first and/or second population of stem cells may be administered to a subject in any pharmaceutically or medically acceptable manner, including by injection or transfusion. The cells or supplemented cell populations may contain, or be contained in any pharmaceutically-acceptable carrier. For example, pharmaceutical compositions of the present invention may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration. The first and/or second population of stem cells may be carried, stored, or transported in any pharmaceutically or medically acceptable container, for example, a blood bag, transfer bag, plastic tube or vial.

A cell composition of the present invention should be introduced into a subject, preferably a human, in an amount sufficient to achieve tissue repair or regeneration, or to treat a desired disease or condition. Preferably, at least about 2.5×10⁷ cells/kg, at least about 3.0×10⁷, at least about 3.5×10⁷, at least about 4.0×10⁷, at least about 4.5×10⁷, or at least about 5.0×10⁷ cells/kg is used for any treatment, either in the first cell population, the second population, or a combination of the first and second population of stem cells. Where cord blood from several donors is used, the number of cord blood stem cells introduced into a subject may be higher. Where the second population of cells is enriched for ASPC, the number of nucleated cells per kg necessary to facilitate or accelerate engraftment may be fewer than 2.5×10⁷ cells/kg. Thus, the methods of the invention may decrease the number of transplanted cells necessary for hematologic recovery. This method is particularly useful when the number of cells available for transplant (e.g., from umbilical cord blood) is limited.

When “therapeutically effective amount” is indicated, the precise amount of the compositions of the present invention to be administered can be determined by an art worker with consideration of a subject's age, weight, tumor size, extent of infection or metastasis, and condition of the subject. The cells can be administered by using infusion or injection techniques that are commonly known in the art.

### VII. Adjuvant Therapy

In accordance with the use of first and second population of stem cells in the methods of the invention, one may also treat the host to reduce immunological rejection of the donor cells, such as those described in U.S. Pat. No. 5,800,539, issued Sep. 1, 1998; and U.S. Pat. No. 5,806,529, issued Sep. 15, 1998, both of which are incorporated herein by reference.

In certain embodiments of the present invention, the cells of the present invention are administered to a subject following treatment with an agent such as myeloablative (high dose) chemotherapy, chemotherapy, radiation, immunosuppressive agents, such as antithymocyte globulin (ATG), busulfan, IVIG, melphalan, methylprednisolone, cyclosporin, azathioprine, methotrexate, mycophenylate, and FK506, antibodies, or other immunoablative agents such as CAMPATH, anti-CD3 antibodies or other antibody therapies, cytoxin, fludaribine, cyclosporin, FK506, rapamycin, mycophenylic acid, steroids, FR901228, cytokines, and localized or total body irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993; Isoniemi (supra)). In a further embodiment, the cell compositions of the present invention are administered to a subject in conjunction with (e.g. before, simulataneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g. Rituxan. For example, in one embodiment, subjects may undergo standard treatment with high dose chemotherapy followed by stem cell transplantation. Following the transplant, subjects receive an infusion of the two cell populations described herein.

The dosage of the above treatments to be administered to a subject will vary with the precise nature of the condition being treated and the recipient of the treatment. The scaling of dosages for human administration can be performed according to art-accepted practices.

### VIII. Monitoring Therapeutic Response

Methods for monitoring therapeutic response in subjects include assessment of one or more of overall and event-free survival, platelet engraftment, ANC engraftment, relapse of disease, or the like, in a subject. The response to treatment can be compared to an appropriate control. Methods for monitoring these responses are well known in the art and exemplified herein.

For the purposes of the present invention, a “subject” refers to an individual that has been administered the cell preparations of the invention. The subject can be a human, a non-human primate, a laboratory animal, or the like, but preferably is a human. A “control” can include an individual (or group of individuals) that is (are) untreated, sham treated (e.g., the individual is treated with a first and second cell population in which one or both populations do not contain the cell preparations described herein), treated with a similar or distinct method for improving engraftment and/or improving therapeutic response to stem cell transplantation, or treated with a cell preparation that has not been enriched for one or more of the stem cell markers described herein, depending on the nature of the observation. For example, if one wishes to compare the therapeutic response of a subject that has been treated with a second cell population that has been enriched for ALDH^{br} cells, an appropriate control may include the therapeutic response of a subject whose second cell population has not been enriched for ALDH^{br} cells, or may include the therapeutic response of a subject whose second cell population has been enriched for an alternative cell surface marker, such as CD34. Alternatively, controls can be historical controls. For example, the response of the subject to the methods of the invention can be compared to the response seen in previously studied populations of subjects undergoing similar or distinct procedures for modulating engraftment and/or improving therapeutic response to stem cell transplantation.

In some embodiments, the methods of the present invention result in a decrease of incidence and/or severity of grade III and/or grade IV acute graft versus host disease (GvHD), in part by eliminating T cell populations. This elimination from the stem cell population of the invention can be expected to reduce the incidence and severity of GvHD in recipients of allogeneic transplants. See, for example, Ho and Soiffer (2001) Blood 98:3192. GvHD occurs when donor T-cells react against antigens on normal host cells causing target organ damage, which often leads to death. The principal target organs of GvHD are the immune system, skin, liver and intestine.

There are two kinds of GvHD: acute and chronic. Acute GvHD appears within the first three months following transplantation. Signs of acute GvHD include a reddish skin rash on the hands and feet that may spread and become more severe, with peeling or blistering skin. GvHD is ranked based on its severity: stage (or grade) 1 is mild, stage (or grade) 4 is severe. Chronic GvHD develops three months or later following transplantation. The symptoms of chronic GvHD are similar to those of acute GvHD, but in addition, chronic GvHD may also affect the mucous glands in the eyes, salivary glands in the mouth, and glands that lubricate the stomach lining and intestines.

Following administration of the cell populations described herein, the subject may be monitored for levels of malignant cells, i.e., for evidence of minimal residual disease. Such monitoring may comprise subject follow-up for clinical signs of relapse. The monitoring may also include, where appropriate, various molecular or cellular assays to detect or quantify any residual malignant cells. For example, in cases of sex-mismatched donors and recipients, residual host-derived cells may be detected through use of appropriate sex markers such as Y chromosome-specific nucleic acid primers or probes. In cases of single HLA locus mismatches between donors and recipients, residual host cells may be documented by polymerase chain reaction (PCR) analysis of Class I or Class II loci that differ between the donor and recipient. Alternatively, appropriate molecular markers specific for tumor cells can be employed. For example, nucleic acid primers and/or probes specific for the bcr/abl translocation in chronic myelogenous leukemia, for other oncogenes active in various tumors, for inactivated tumor suppressor genes, other tumor-specific genes, or any other assay reagents known to be specific for tumor cells, may be employed. Any of these or functionally comparable procedures may be used to monitor the subject for evidence of residual malignant cells. In one embodiment, the methods of the present invention result in at least about a 10%, at least about a 15%, at least about a 20%, about a 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, or at least about a 100% decrease in the presence of malignant cells when compared to a control.

Treatment of a subject according to the methods of the present invention may be considered efficacious if the disease, disorder or condition is measurably improved in any way. Such improvement may be shown by a number of indicators. Measurable indicators include, for example, detectable changes in a physiological condition or set of physiological conditions associated with a particular disease, disorder or condition (including, but not limited to, blood pressure, heart rate, respiratory rate, counts of various blood cell types, levels in the blood of certain proteins, carbohydrates, lipids or cytokines or modulated expression of genetic markers associated with the disease, disorder or condition). Treatment of an individual with the stem cells or supplemented cell populations of the invention would be considered effective if any one of such indicators responds to such treatment by changing to a value that is within, or closer to, the normal value. The normal value may be established by normal ranges that are known in the art for various indicators, or by comparison to such values in a control. In medical science, the efficacy of a treatment is also often characterized in terms of an individual's impressions and subjective feeling of the individual's state of health. Improvement therefore may also be characterized by subjective indicators, such as the individual's subjective feeling of improvement, increased well-being, increased state of health, improved level of energy, or the like, after administration of the cell populations of the invention. In one embodiment, the methods of the present invention result in at least about a 30%, at least about a 35%, about 40%, about 50%, about 60%, about 70%, about 80%, about 90%, about 100%, about 125%, about 150%, about 175%, about 200%, about 250%, at least about a 300%, or greater improvement in one or more of the clinical indicators described above when compared to a control.

The primary measure of hematologic recovery is neutrophil count. Neutrophils usually constitute about 45 to 75% of all white blood cells in the bloodstream. When the neutrophil count falls below 1,000 cells per microliter of blood, the risk of infection increases somewhat; when it falls below 500 cells per microliter, the risk of infection increases greatly. Without the key defense provided by neutrophils, controlling infections is problematic and subjects are at risk of dying from an infection. Accordingly, in clinical settings, such as transplant settings, the sooner neutrophil counts recover, the sooner a subject can be released from the hospital. Accordingly, any decrease in time that it takes to achieve clinically relevant levels of neutrophils is beneficial to the subject and contemplated herein as acceleration of hematologic recovery. For the purposes of the present invention, neutrophil engraftment is defined as an absolute neutrophil count (ANC) of at least 500 neutrophils/μl. The neutrophil count may be reported as a date that an individual subject (or an average of multiple subjects) reaches the ANC threshold, or a percentage of the subjects having an ANC of 500 neutrophils/μl by a particular day post-transplant, usually around day 42, or the probability that an individual will reach a certain threshold by a certain date. In one embodiment, the methods of the present invention result in neutrophil engraftment on or before day 10, day 11, day 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or on or before day 48. In another embodiment, the day that patients achieve a benchmark ANC count deemed to be normal will be accelerated by 5 days, 6 to 10 days, 11-20 days, or greater than 20 days relative to a control group of patients.

Hematologic recovery can also be measured by a clinically relevant recovery of platelets (as would be recognized by the skilled artisan, there are normally between 150,000-450,000 platelets in each microliter of blood). Thus, any increase in the rapidity of a clinically relevant recovery of platelets is advantageous and contemplated herein. For the purposes of the present invention, platelet engraftment is defined as maintenance of platelet counts of at least 50,000 platelets/μl of blood without transfusion support. The platelet count may be reported as a date that an individual subject (or an average of multiple subjects) reaches the platelet count threshold, or as a percentage of the subjects having (or probability of a subject reaching) a platelet count of at least 50,000 platelets/μl of blood by a particular day post-transplant, usually around day 180. In one embodiment, the methods of the present invention result in platelet engraftment on or before day 50, day 55, day 60, 65, 70, 75, 80, 85, 90, 95, or on or before day 100. In another embodiment, the day that patients achieve a benchmark platelet count deemed to be normal will be accelerated by 5 days, 6 to 10 days, 11-20 days, or greater than 20 days relative to a control group of patients.

In certain embodiments, rapidity in T cell recovery is also an indicator of accelerated hematologic recovery. An indicator of T cell recovery can include response to PHA-induced profileration and/or an increase in the number of CD4+ cells in the subject. The CD4+ counts may be reported as a date that an individual subject (or an average of multiple subjects) reaches a CD4+ count threshold, or as a percentage of the subjects having (or the probability of subject reaching) a threshold CD4+ count by a particular benchmark day post-transplant, usually around day 100. In one embodiment, the methods of the present invention result in T cell counts at day 100 that are at least about 25 to 100% or greater than counts in patients in a control population. In another embodiment, the post-transplant day that a patient achieves a benchmark CD4 count is about 10 to about 20, about 20 to about 30, about 30 to about 40, about 40 to about 50, or greater than 50 days earlier than the day that patients in a control group achieve the same benchmark CD4 count.

A therapeutic response can also be measured in terms of overall and/or event free survival. Event free survival (EFS) is defined as the time from transplantation to the day of the first event. Events are defined as graft failure, autologous reconstitution, relapse, or death. Relapse in leukemic subjects is determined by standard criteria. Tertiary end points include description of the incidence of acute GvHD, and other measures of nonrelapse mortality. GvHD is scored according to standard criteria (Przepiorka et al. (1995) Bone Marrow Transplant. 15: 825-828). In one embodiment, the methods of the present invention result in overall and/or event-free survival that is at least about 30%, at least about 35%, 40%, 50%, 60%, 70%, 80%, 90%, 100%, 125%, 150%, 175%, 200%, 250%, at least about 300%, or greater % improved over controls (e.g., fewer incidences of events (particularly grade III and/or grade IV acute GvHD) reported, increased number of days of survival, and/or higher numbers of patients surviving to a certain date post-transplant when compared to a control population).

Another global measure of therapeutic response is overall survival at 180 days. In this metric, survival in the group of patients transplanted according to the present invention is compared to overall survival in a control group treated by conventional methods. In one embodiment of the invention, patients show an improved overall survival of at least about 5%, of at least about 6-10%, of at least about 11-15%, of at least about 16-20%, or of great than 20% compared to control patients.

The following examples are offered by way of illustration and not by way of limitation.

### EXPERIMENTAL

### Example 1

### Immune Reconstitution After Unrelated Mismatched UCB Transplantation

Immune reconstitution has been evaluated in approximately 100 survivors of UCB transplantation that have been followed for a median of 650 days (range 121-2450 days). The results of this study can be found in Klein et al. (2001) Biol Blood and Marrow Trans 7:454-466. Briefly, functional and immunophenotypic parameters were assayed in engrafted patient's peripheral blood at 3, 6, 9, 12, 24, and 36 months post transplant. Patients were generally maintained on methyprednisolone for the first three months post transplant and cyclosporine for the first year post transplant. Immunizations were reinstituted in the second and third years post transplant. All surviving patients without active chronic GvHD received the full complement of killed and live vaccines per the usual CDC recommendations. Infants and toddlers <2 years of age recovered T-cell immune function as measured by CD4 counts and PHA responses by 6 months post transplant. Children between the ages of 2-12 years recovered similar function by 9-12 months post transplant. In contrast, teenagers and adults recovered immune function by 3 years post-transplant. It appears that the host thymus contributes to immune reconstitute from the UCB graft. The younger the patient and the healthier the thymus (e.g. no exposure to pre-transplant irradiation), the quicker the thymic recovers and contributes to immune reconstitution from the graft. Children normalized by 1 year post transplant, while adults approached the lower limit of normal for age by 3 years post transplant. In the interim, adults reconstituted T-cells by peripheral mechanisms. Those patients with earlier immune reconstitution faired better with transplant overall. They were less likely to develop an opportunistic infection in the first 2-4 months post transplant. The patients in this category had superior survival. Percent CD4 cells was the best predictor of lack of opportunistic infection (p=<0.001).

### Example 2

### Clinical Results of UCB Transplantation in Pediatric Patients with Inborn Errors of Metabolism

Recent results from the Cord Blood transplantation Study (COBLT), a multi-institutional, prospective NIH-sponsored trial of unrelated donor cord blood transplantation have further advanced the field of UCBT. See, Kurtzberg et al. (2005) Biology of Blood and Marrow Transplantation 11(2):2 (abst 6); Kurtzberg et al. (2005) Biology of Blood and Marrow Transplantation 11(2):82(Abst 242).

A different strata of the COBLT study evaluated the efficacy of cord blood transplantation in 69 children with inborn errors of metabolism, augmenting prior and pending reports results of UCBT in babies with Infantile Krabbe Disease and Hurler Syndrome (MPS I). A common protocol was used for the preparative regimen (busulfan, cyclophosphamide, ATG) and GvHD prophylaxis (cyclosporine, steroids). Patients with MPS 1-V (n=36, 20 previously reported), globoid cell leukodystrophy (Krabbe Disease, n=16), adrenoleukodystrophy (n=8), metachromatic leukodystrophy (n=6) and Tay Sachs Disease (n=3) with a median age of 1.8 years (range 0.1-11.7 years) and a median weight of 12.3 kg (range 3.9-42.3 kg) were transplanted with partially HLA-mismatched unrelated donor cord blood delivering a median of 8.7×10e7 nucleated cells/kg (range 2.8-38.8 cells/kg) selected from COBLT (83%) or other (17%) banks. CBUs were screened for enzyme activity to prevent use of carrier donors. Sixty-four percent of patients were male and 77% were Caucasian. Nearly half the patients (48%) received a UCB units matching at 4/6 HLA loci as measured by low resolution typing at HLA Class I A&B and high resolution typing at HLA Class II DRB1.

The cumulative incidence of neutrophil engraftment (ANC 500/uL with 90% donor chimerism by day 100) was 78%, occurring in a median of 26 days. The cumulative incidence of acute Grades II-IV GvDH was 46%. The probability of survival at 180 days and 1 year was 80 and 72%, respectively. Levels of HLA disparity between recipient and donor did not influence engraftment, GvHD or overall survival. The surviving patients with MPS, TSD, GLD, and MLD all stabilized and/or gained skills post transplant. Three of 8 patients with ALD, all of whom had mild to moderate clinical symptoms at the time of referral for transplant, experienced disease progression with neurologic deterioration before stabilization. Outcomes in babies with the severe phenotype of Hurler Syndrome (Kurtzberg, 2005, supra and Dexter et al. (1977) J Cell Physiol 91:335-344) and newborns with Krabbe Disease (Gartner et al. (1980) Gartner Proc Natl Acad Sci 77:4756-4759) transplanted before the onset of symptoms were unprecedented with the vast majority of patients having normal intelligence quotients for age. The younger the age at transplant and the earlier in the course of the disease, the better the overall outcome. Therefore, it is clear that cord blood transplantation offers a rapidly available donor source for early treatment of infants, toddlers and children with inborn errors of metabolism

### Example 3

### Prepurification Steps to Enrich for ALDH^{br} UCB Cells

The cord blood unit selected for transplantation is stored in a 2 compartment cryopreservation bag (20%/80% split) in a total of 25 ml of cells, hespan and 10% DMSO. On day −5 before transplant, the 20% (5 ml) fraction is removed from liquid nitrogen (procedure 5D.160.01), and thawed in a 37 degree C. waterbath to a slushy consistency. Dextran/Albumin is added to dilute to 4×the initial volume, the cells are washed, pelleted and resuspended in ALDESORT® assay buffer/100U/ml DNase I (Aldagen, Inc., Durham, N.C.). Red blood cell to white blood cell ration is adjusted to <1×10e8 cells/ml and the cells are lineage depleted with EASYSEP® (StemCell Technologies) anti-glycophorin A and CD14 cocktails to label cells. The labeled cells are mixed with EASYSEP® magnetic nanoparticles and incubated at room temperature for 10 minutes. The sample is then exposed to the EASYSEP® magnetic which will remove lineage positive cells. The residual lineage depleted cells are gently aspirated into a conical tube. RBC: WBC ratio is checked and must be <1:10. If it is higher, the EASYSEP® depletion is repeated.

### Example 4

### Isolation of ALDH^{br} UCB Cells by High Speed FACS Sorting

The lineage depleted cells are stained with activated ALDESORT® reagent and incubated at 37 degrees C. for 15 minutes. The reaction is stopped, controls are prepared and the ALDH^{br} cells are isolated by high speed flow sorting on the FACSAria sorter (BD Biosciences). Methods for isolating ALDH^{br} cells are more fully described in Storms et al., 1999, supra and PCT Publication No. WO 2005/083061, both of which are herein incorporated by reference in their entirety. The cells may be frozen or immediately infused as described below.

### Example 5

### UCB Thawing and Infusion for the Conventional, Unmanipulated Graft (First Cell Population)

Bags of UCB are thawed in the laboratory using sterile technique under a hood. The UCB is thawed in a 37° C. waterbath, and diluted by 1:1 volume using a 5% albumin/dextran solution [albumin 25% (12.5 gms/50 ml) 25 gms in 500 ml dextran] to preserve cell viability. The 5% albumin/dextran solution is added slowly to the thawed UCB using transfer bags with stopcocks and mixed gently. The thawed and diluted UCB is next weighed and centrifuged (2000 rpm×20 min at 4° C.). Specimens are obtained for cell count and viability, culture, clonogenic assays, and phenotype. Supernatant containing DMSO and the albumin/dextran solution is removed, and the UCB pellet resuspended again by a 1:1 volume using a 5% albumin/dextran solution. The UCB is labeled with patient identification information and transferred to the bedside for infusion. The UCB is infused via the patient's central venous catheter at a rate of 1-3 ml/min. UCB is infused without an in-line filter and is not irradiated. If the patient develops chest tightness or other symptoms, a brief rest (1-2 minutes) may be required before proceeding with the remainder of the infusion. If a large volume of UCB (>15 ml/kg) is to be infused, half the UCB may be infused, followed by a 30 minute rest period, and then infusion of the remainder of the UCB. Vital signs are taken every 15 minutes until 2 hours after completion of the infusion. Hydration (2.5-3.0 ml/kg/hr) is maintained for 12 hours after UCB infusion is completed. Furosemide (0.5-1.0 mg/kg/dose) is given if volume overload or decreased urine output occurs.

### Example 6

### Thawing, Sorting, and Infusion of the ALDH^{br} Cells (Second Population)

The UCB cells are thawed and ALDH^{br} sorted (if not sorted prior to freezing). On day 0, transplant day, approximately 4 hours after infusion of the conventional UCB graft, the ALDH^{br} UCB cells are harvested, counted, checked for viability and gram stain, connected to the infusion set and transported to the bone marrow transplant unit for infusion.

### Example 7

### Conditioning of Patients with Malignant Conditions

Standard cytoreduction for patients with ALL undergoing allogeneic BMT includes cyclophosphamide (100-200 mg/kg) and total body irradiation (TBI, 1,000-1440 cGy). With these regimens, event-free survival rates can be achieved in 20-45% of children and 20% of adults with ALL in 2nd remission, and up to 60% of patients with ANLL undergoing matched-related allogeneic BMT. With subsequent remissions, event-free survival decreases with only 8% of patients cured when transplanted in relapse. ATG is used for additional immunosuppressive therapy; methylprednisolone is substituted if patients cannot tolerate ATG.

The rates of engraftment, GvHD, relapse and survival from the COBLT study (Klein et al. 2001, supra; Martin et al, 2006, supra) are used as historical controls to benchmark the success of the transplant.

### Example 8

### Conditioning of Patients with Non-Malignant Conditions

Standard cytoreduction for patients with non-malignant conditions undergoing allogeneic BMT includes busulfan 16 mg/kg over 4 days (adjusted for pediatric patients to dosing per m2 and followed with targeted levels with first dose PK), cyclophosphamide 200 mg/kg over 4 days and ATG 90 mg/kg over 3 days. Engraftment rates with unrelated donor umbilical cord blood using this regimen ranges between 80-90%. TBI is avoided to minimize late adverse events such as growth retardation, endocrine failure, cognitive deficits, chronic lung disease or cardiomyopathy.

### Example 9

### Evaluation of Engraftment

A peripheral blood sample is tested on or about days +30, 60 and 100 for chimerism. A bone marrow aspirate and biopsy for cellularity and donor chimerism is performed between days 41-44 if the patient has not demonstrated neutrophil recovery by this time. Platelet counts, ANC, GvHD, and various other clinical indicators of successful engraftment are then evaluated as known in the art.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims and list of embodiments disclosed herein. Although specific terms are employed herein, they are used in a generic and descriptive sense only and not for purposes of limitation.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

That which is claimed:

1. A method of reconstituting blood tissue in a subject in need thereof, said method comprising introducing into said subject a first and a second population of cells, wherein said second population of cells is introduced between 2 and 24 hours after the first population of cells, and wherein at least the second population is capable of engraftment.

2. The method of claim 1, wherein said second population of cells is introduced about 4 hours after the first population.

3. The method of claim 1, wherein both the first and the second population of cells are capable of engraftment.

4. The method of claim 1, wherein the first and second populations of cells are derived from a source selected from the group consisting of umbilical cord blood, bone marrow, and mobilized peripheral blood.

5. The method of claim 4, wherein the first and second populations of cells are derived from the same source.

6. The method of claim 4, wherein the first and second populations of cells are derived from different sources.

7. The method of claim 1, wherein said subject is in need of hematopoietic reconstitution following bone marrow ablation.

8. The method of claim 1, wherein at least the second population of cells is enriched for adult stem and progenitor cells.

9. The method of claim 8, wherein the second population of cells is introduced about 4 hours after the first population.

10. A method of restoring hematologic function following myeloablative treatment in a subject having cancer comprising introducing into said subject a first and a second population of cells, wherein said second population of cells is introduced between 2 and 24 hours after the first population of cells, and wherein at least the second population is capable of engraftment.

11. The method of claim 10, wherein at least the second population of cells is enriched for adult stem and progenitor cells.

12. The method of claim 10, wherein said subject is in need of treatment for sequelae related to cancer therapy.

13. A method for potentiating engraftment of a stem cell population in a subject in need thereof comprising introducing into said subject a first and a second population of cells, wherein said second population of cells is introduced between 2 and 24 hours after the first population of cells, and wherein at least the second population is capable of engraftment.

14. The method of claim 13, wherein at least the second population of cells is enriched for adult stem and progenitor cells.

15. The method of claim 13, wherein the time to neutrophil engraftment in the subject is shortened compared to the time to neutrophil engraftment in a control subject.

16. The method of claim 13, wherein the time to platelet engraftment is shortened compared to the time to platelet engraftment in a control subject.

17. A method of restoring hematologic function following myeloablative treatment in a subject having a genetic disorder comprising introducing into said subject a first and a second population of cells, wherein said second population of cells is introduced between 2 and 24 hours after the first population of cells, and wherein at least the second population is capable of engraftment.

18. The method of claim 17, wherein at least the second population of cells is enriched for adult stem and progenitor cells.

19. A method of restoring bone marrow stem or progentitor cell activity following myeloablative treatment in a subject having cancer comprising introducing into said subject a first and a second population of cells, wherein said second population of cells is introduced between 2 and 24 hours after the first population of cells, wherein at least the second population is an enriched ALDH^{br} stem cell population.

20. A method of restoring bone marrow stem or progentitor cell activity following myeloablative treatment in a subject having a genetic disorder comprising introducing into said subject a first and a second population of cells, wherein said second population of cells is introduced between 2 and 24 hours after the first population of cells, wherein at least the second population is an enriched ALDH^{br} stem cell population.
